(19) United States
(12) Patent Application Publication
(10) Pub. No.: US 2022/0408810 A1
(43) Pub. Date: Dec. 29, 2022

(54) VAPE PEN AND VAPING FLUID DELIVERY CONTAINER PROTECTION DEVICE AND DECORATIVE COVER

(71) Applicant: Vitamin Leaf LLC, Voorhees, NJ (US)

(72) Inventor: Charles Maimone, Voorhees, NJ (US)

(21) Appl. No.: 17/774,852

(22) PCT Filed: Nov. 3, 2020
(86) PCT No.: PCT/US2020/058724

§ 371 Date: May 5, 2022

Related U.S. Application Data
(60) Provisional application No. 62/931,070, filed on Nov. 5, 2019.

(51) Int. Cl. A24F 40/42; A24F 40/10

(52) U.S. Cl. CPC A24F 40/42; A24F 40/10

(57) ABSTRACT

A protective sleeve (100) is provided for vape fluid cartridges or ampules (130). Open ends (103, 104) facilitate insertion of a vape fluid cartridge (130). One or more apertures (120A, 120B) may be provided to permit viewing of cartridge fluid level. The sleeve (100) may be formed from an elastic material, such as silicone. An elevated section (110) may be provided to inhibit rolling and/or permit ornamentation.

### TECHNICAL FIELD

Embodiments of the present disclosure relate in general to vape pens, and more particularly, to a protective and/or insulative device for vaping fluid ampules and vape pens.

### BACKGROUND

Vaping is becoming increasing popular for recreational and/or therapeutic purposes. Typically, vaping is performed using a battery-powered device such as a vape pen (some of which can be referred to as an e-cigarette). In use, the vape pen vaporizes liquids containing flavorings, active substances (such as nicotine, THC or pharmaceuticals) or other substances, to facilitate inhalation by the user.

Vape fluid is commonly sold and utilized within cartridges (also referred to as ampules), which can be interchangeably attached to, and removed from, a vape pen for contents delivery. Users may have a variety of different cartridges, often containing different substances to enable the users to select between different vaping fluids using a common vape pen. However, vape fluid cartridges are commonly made from glass (e.g. for chemical non-reactivity) or plastic. Such cartridges can be fragile, and prone to breakage when dropped. Cartridges or ampules are typically smooth in texture and relatively small in size, both also increasing risk of droppage and breakage. Moreover, many vape fluid cartridges are cylindrical in shape, making them highly prone to rolling when placed on a tabletop or other flat surface. Meanwhile, vaping fluids can be quite expensive, particularly for fluids containing medicinal and/or psychoactive compounds. Thus, breakage of a cartridge and ensuring loss of the cartridge contents can be costly for a user.

Additionally, vape pens themselves are often compact devices, to facilitate easy storage within a user's pocket or elsewhere. However, they can also be costly devices that are fragile, and prone to dropping due to frequent handling.

### SUMMARY

A protective sleeve is provided for a cartridge or ampule containing fluids, such as a vape fluid cartridge. In an embodiment, the sleeve may have open ends for insertion of a vape fluid cartridge into an internal cavity of the sleeve, which may be cylindrical. An aperture, such as one or more elongated gaps, slots or channels, may be provided along the length of the sleeve, exposing at least a portion of a vape fluid cartridge installed within the internal cavity, whereby a user may visually evaluate a level of fluid remaining within a transparent or translucent cartridge. The aperture may be formed from one or more gaps in material, or from one or more portions of transparent or translucent material (i.e. windows) within an otherwise opaque sleeve.

Various structures may be provided in an exterior surface of the sleeve to improve a user's ability to grip the sleeve and/or inhibit rolling. An elevated section may extend outward from an exterior surface of the sleeve. The elevated section may form a flat surface, which may be generally rectangular in shape and which may permit ornamentation, such as added text or graphic elements. One or more protrusions, such as semi-spherical bumps, may be provided on the sleeve outer surface, also to facilitate gripping and/or inhibit rolling.

The sleeve may be formed from materials such as silicone, urethane, polyurethane, thermoplastic, polyethylene or elastomer. The sleeve material will preferably provide elasticity for impact resistance and secure fit, while also having a slip-resistant outer surface.

In yet other embodiments, the sleeve may be formed from a single continuous length of flat material wrapped in a spiral pattern to form a cylindrical cavity within in. The wrap sleeve may be secured to a vape cartridge via adhesive, or via an inherent spring bias. Gaps may be provided between adjacent sections of the spiral wrap sleeve to permit viewing of fluid levels within a contained vape cartridge.

These and other aspects will become evident in light of the drawings and text provided herein.

### BRIEF DESCRIPTION OF THE FIGURES

FIG. 1 is an upper left side perspective view of a protective sleeve, in accordance with one embodiment.

FIG. 2 is a right side perspective view of the protective sleeve.

FIG. 3 is a left side view of the protective sleeve.

FIG. 4 is a front view of the protective sleeve.

FIG. 5 is a rear view of the protective sleeve

FIG. 6 is a rear view of the protective sleeve, installed on a fluid cartridge.

FIG. 7 is a rear view of a protective sleeve, in accordance with another embodiment.

FIG. 8 is a rear view of a protective sleeve, in accordance with a further embodiment.

FIG. 9 is a perspective view of a protective sleeve having raised bumps, installed on a cartridge.

FIG. 10 is a perspective view of a protective sleeve having an outer surface with polygonal cross-section.

FIGS. 11 and 12 are perspective views of a protective sleeve during wrap installation on a cartridge, in accordance with another embodiment.

### DETAILED DESCRIPTION OF THE DRAWINGS

While this invention is susceptible to embodiment in many different forms, there are shown in the drawings and will be described in detail herein several specific embodiments, with the understanding that the present disclosure is to be considered as an exemplification of the principles of the invention to enable any person skilled in the art to make and use the invention, and is not intended to limit the invention to the embodiments illustrated.

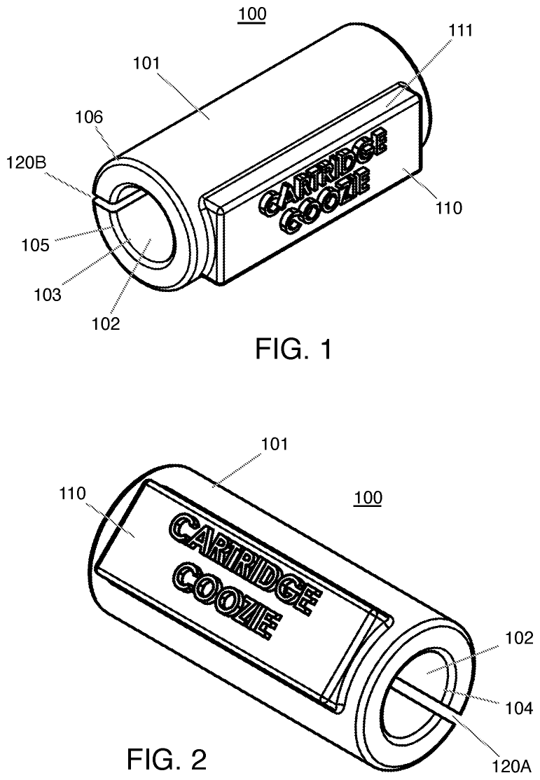

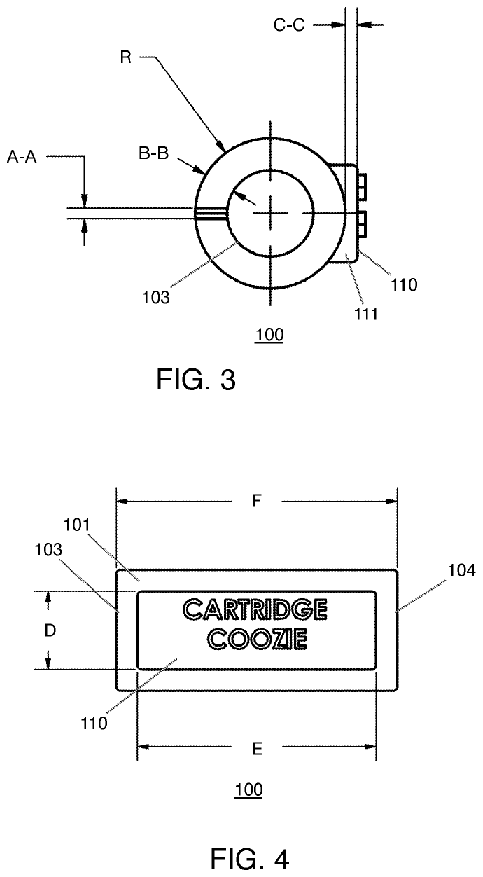

FIG. 1 is an upper left perspective view of a protective sleeve 100, in accordance with one embodiment. FIG. 2 provides a right side perspective view of sleeve 100. FIG. 3 provides a left side elevation of sleeve 100, and FIG. 4 is a rear view of sleeve 100.

Sleeve 100 is generally cylindrical in shape, having an exterior surface 101 and smooth interior surface 102. First end 103 and second end 104 each feature an opening providing access to the interior of sleeve 100. Interior end chamfer 105 facilitates insertion and removal of an ampule or cartridge from first end 103, while exterior chamfer 106 provides for comfortable handling of sleeve 100 (and, when inserted, an ampule or cartridge). While sleeve 100 may include apertures and/or elevated portions, as described hereinbelow, preferably at least a portion of exterior surface 101 extends continuously from first end 103 to second end 104.

Preferably, sleeve 100 may be formed from a material that provides reduced slippage as compared to a cartridge or ampule on which the sleeve may be installed, as well as shock absorption to reduce risk of damage to a cartridge from droppage or other impact. Materials that may be utilized include silicone, urethane, polyurethane, thermoplastic, polyethylene, elastomer or similar materials. Material may be selected to provide suitable elasticity, durometer measurement, and tear resistance to provide a layer of protection that is sufficiently thin to avoid interference with an e-cigarette or vaping device when installed on a cartridge in use.

Thermal conditions may also be important with respect to vaping devices. Vaping devices typically utilize thermal energy to heat vaping fluids and encourage vaporization during inhalation. However, as a result, in some arrangements, vape fluid cartridges and/or vape devices themselves may become uncomfortably warm to the user's touch. In addition to providing improved grip and impact resistance, protective sleeve embodiments described herein may provide thermal insulation for a user. Additionally, it is possible that very cold ambient temperatures may inhibit efficient operation of a vaping device, such as in the event that a device is exposed to winter outdoor conditions for an extended period during use. In such circumstances, thermal insulative effects of the sleeve may promote efficient operation of the device and/or inhibit freezing of cartridge contents.

The exterior surface of sleeve 100 includes an elevated section 110, extending outwards from exterior surface 101 on one side of sleeve 100. In some embodiments, elevated section 110 forms a flat outward-facing surface, facilitating the inclusion of ornamentation such as text, logos, symbols, or the like. Such ornamentation may be embossed, debossed, engraved, and/or printed on elevated section 110. Moreover, side surfaces 111 of elevated section 110 provide a structure for a user to more affirmatively grip sleeve 100 (and its contents, when installed). Side surfaces 111 also inhibit rolling of sleeve 100 (and its contents, when installed) when sleeve 100 (and its contents) are placed on a flat or relatively flat surface, such as a tabletop.

Particularly in circumstances where a protective sleeve may be used with an ampule or cartridge that enables a user to view the contents within it (e.g. an ampule in which all or a portion is translucent or transparent, such as may be formed from glass or plastic), it may be desirable to provide a protective sleeve that does not preclude the user from viewing at least portions of the ampule within, thereby, e.g., enabling a user to assess the level of fluids remaining. To that end, one or more apertures may be provided in the protective sleeve, exposing the interior of the sleeve to viewing by a user.

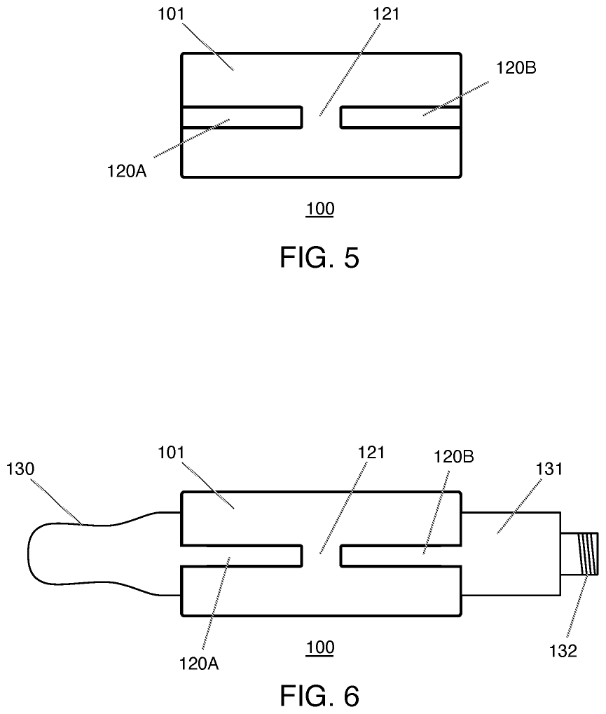

For example, as illustrated in the rear view of FIG. 5, sleeve 100 includes slots 120A and 120B, enabling a user to view portions of an ampule or cartridge contained within the sleeve 100 to assess the level of fluid remaining. Central portion 121 of sleeve 100 separates slots 120A and 120B, maintaining a continuous outer surface 101 around the circumference of sleeve 100. Utilization of a central portion 121 and continuous circumference permits use of a variety of materials for sleeve 100, including relatively soft, elastic materials such as silicone, while still providing a secure fit for sleeve 100. While central portion 121 may cause the complete occlusion of a small portion of an ampule or cartridge installed within sleeve 100, the occlusion would still enable a user to determine an approximate level of cartridge contents, and any imprecision resulting from the occlusion would occur when the fluid level is roughly at its mid-point—a state at which precise fill level may be less critical.

FIG. 8 is a rear view of sleeve 100, as installed on an exemplary vape fluid cartridge 130. During installation, cartridge 130 is inserted into one of sleeve ends 103 or 104. Inner surface 102 of sleeve 100 slides over an outer surface 131 of cartridge 130. Preferably, cartridge 130 has a diameter that is the same as, or slightly larger than, a diameter of sleeve inner surface 102, such that elastic properties of sleeve 100 secure sleeve 100 onto cartridge 130 during normal use. Sleeve 100 maintains exposure of threaded end 132 of cartridge 130, such that cartridge 130 may be installed and utilized with sleeve 100 in place. Meanwhile, to the extent that at least a portion of cartridge outer surface 131 is transparent or translucent to provide visibility of fluids therein, a cartridge fluid level may be monitored via apertures 120A and 120B.

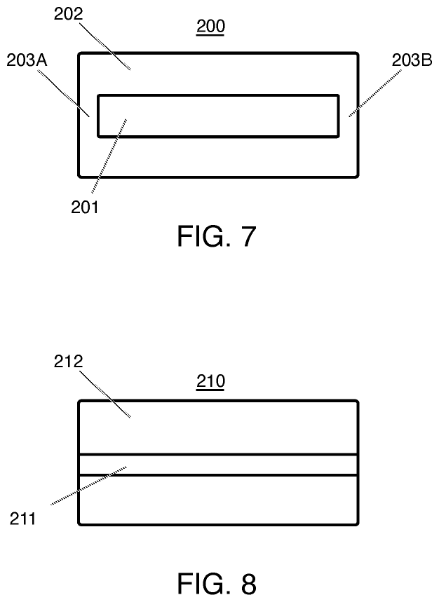

Alternative structures for providing user perception of cartridge fluid level may be utilized. For example, FIG. 7 illustrates a sleeve 200 in rear view, in an alternative embodiment. A single, central aperture 201 is provided within sleeve 200 outer surface 202, in order to provide a user with visibility to a cartridge contained therein, while end portions 203A and 203B provide structure continuity to ensure a tight fit around a contained cartridge, even when the sleeve is formed from a relatively elastic material. FIG. 8 illustrates yet another alternative embodiment, in which sleeve 210 is comprised of a relatively more rigid material, and aperture 211 in sleeve outer surface 212 may extend across the entire length of the sleeve 200, providing unobstructed cartridge fill level perception while relying upon e.g. a relatively high structural rigidity of the sleeve material to maintain a tight fit around an enclosed cartridge.

In yet other embodiments, structures other than apertures may be provided to enable a user to view ampule contents. For example, a sleeve formed primarily from an opaque material (e.g. colored silicone) may include one or more portions formed from a different material that is transparent or translucent, to enable viewing of a cartridge installed therein. Thus, in the embodiment of FIG. 7, instead of providing a physical aperture 201 (i.e. slot), aperture 201 may provide an optical aperture such as a window formed from transparent plastic inset within an otherwise-opaque sleeve outer surface 202.

FIGS. 3 and 4 illustrates exemplary dimensions, in accordance with one embodiment of sleeve 100. Slots 120 may be approximately 1 mm in width (dimension A-A). The thickness of sleeve 100 (dimension B-B) may preferably be 3 mm, about 3 mm, or ranging from 2.5 mm to 3.5 mm. The outer radius R of cylindrical portions of sleeve 100 may be 7.1 mm, about 7.1 mm or ranging from 6.6 mm to 7.6 mm. Elevated section 110 may have an outwardly-facing surface that is rectangular in shape, with width of about 9.19 mm (dimension D) and length of about 28 mm (dimension E). The extent to which elevated section 110 extends outwardly from exterior surface 101 (dimension C-C) may be 1.16 mm, exclusive of any raised text or other graphic content that may be provided thereon. The overall length of sleeve 100 may be about 33 mm (dimension F).

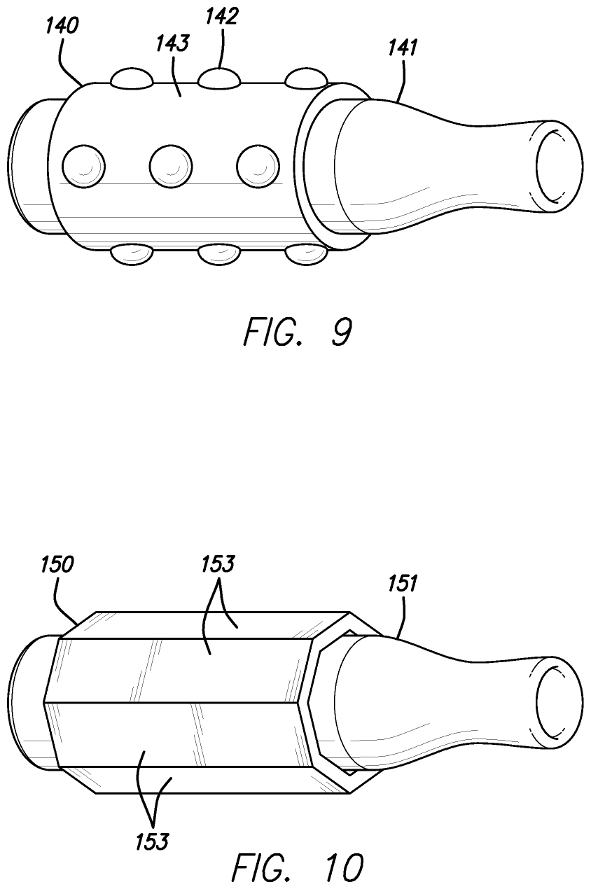

While sleeve 100 is illustrated as some or all of its outer and inner surfaces formed primarily cylindrically (i.e. cylindrically with function discontinuities such as apertures 120A and 120B, or elevated portion 110), it is contemplated and understood that other arrangements may be utilized in other embodiments. For example, FIG. 9 illustrates an alternative embodiment in which sleeve 140 is installed on cartridge 141. Sleeve 140 features numerous raised bumps 142 distributed around sleeve outer surface 143. Raised bumps 142 may be, e.g., semi-spherical in shape, and provide a user with enhanced grip, enhanced inhibition of rolling when set on a flat surface (particularly to the extent sleeve 140 does not include an elevated portion akin to elevated portion 110), and/or improved shock absorption in the event of droppage.

In yet other embodiments, the outer and/or inner surfaces of the sleeve may be formed having differing cross-sectional shapes. For example, FIG. 10 illustrates sleeve 150 installed on cartridge 151. Sleeve 150 includes an exterior perimeter or circumference that has a polygonal (e.g. hexagonal) cross-section, with a plurality of flat exterior surfaces 153 extending over the length of sleeve 150. Ridges formed where flat surfaces 153 join enhance each of grasping, while the relative flatness of surfaces 153 inhibit rolling when placed on a flat surface such as a table.

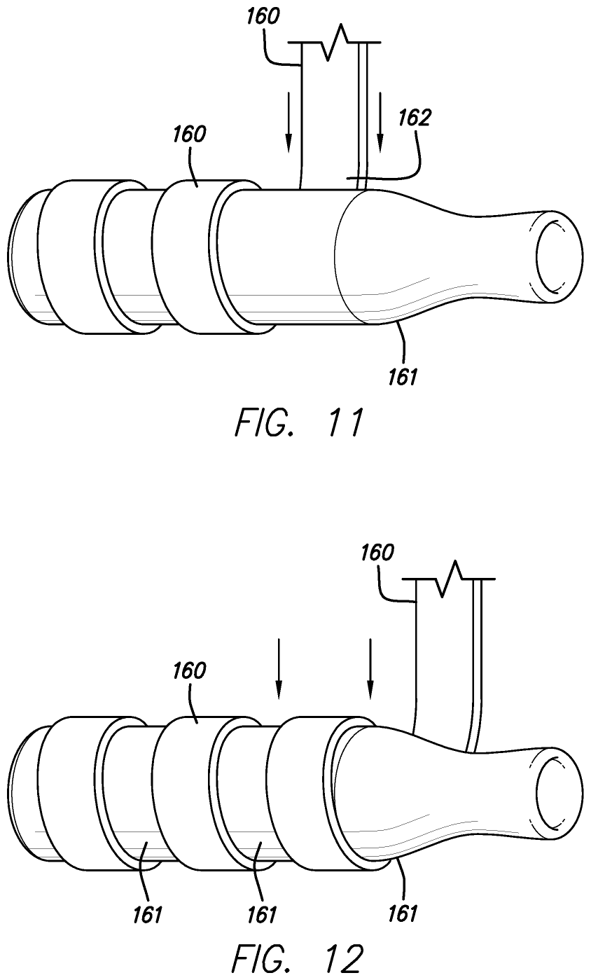

A protective sleeve may also be installed around a cartridge via a wrapping technique. FIGS. 11 and 12 illustrate such an embodiment. A wrap sleeve 160 is formed from an extended length of flat material. Wrap sleeve 160 may then be wrapped in a spiral pattern multiple times around an outer circumference of cartridge 161. When installed, wrap sleeve 160 provides additional texture to facilitate user grasping of cartridge 161, while also providing shock absorption and impact resistance. FIG. 11 illustrates an initial stage of wrapping of sleeve 160 around cartridge 161, while FIG. 12 illustrates further wrapping of sleeve 160 around cartridge 161.

Various structures may be utilized in order to secure wrap sleeve 160 about cartridge 161. In some embodiments, a tacky adhesive may be applied to underside 162 of wrap sleeve 160, in order to secure wrap sleeve 160 onto an outer surface of cartridge 161. In other embodiments, wrap sleeve 160 may be formed from material spring-biased into a rolled configuration, in which case a user may apply force to wrap sleeve 160 to unroll it for installation onto cartridge 161, with the spring-bias of wrap sleeve 160 holding itself in place on cartridge 161 after installation.

In some embodiments, wrap sleeve 160 may be installed on cartridge 161 with spacing or gaps 164 between rotations of wrap sleeve 160, thereby providing an aperture through which the contents of cartridge 161 may be viewed.

While certain embodiments of the invention have been described herein in detail for purposes of clarity and understanding, the foregoing description and Figures merely explain and illustrate the present invention and the present invention is not limited thereto. It will be appreciated that those skilled in the art, having the present disclosure before them, will be able to make modifications and variations to that disclosed herein without departing from the scope of the invention or appended claims.

1. A protective sleeve for a vape fluid cartridge, comprising:
a sleeve having a first open end and a second open end for insertion of a vape fluid cartridge into a cylindrical internal cavity; and
an aperture within a side surface of the sleeve, exposing at least a portion of a vape fluid cartridge within the cylindrical cavity.

2. The protective sleeve of claim 1, wherein the aperture comprises a gap in an exterior surface of the sleeve, and at least a portion of the exterior surface of the sleeve extends continuously from the first end to the second end.

3. The protective sleeve of claim 2, wherein the aperture comprises one or more elongated channels formed in the sleeve surface, the elongated channels collectively facilitating perception of a fluid level of a cartridge inserted within the sleeve.

4. The protective sleeve of claim 2, where the aperture comprises:
a first elongated channel extending from a first end of the sleeve towards the sleeve center;
a second elongated channel extending from a second end of the sleeve towards the sleeve center; and
a central portion of the sleeve, separating the first elongated channel from the second elongated channel.

5. The protective sleeve of claim 2, where the aperture comprises an elongated channel having a first channel end proximate a first end of the sleeve and separated from the first end of the sleeve by a first end portion, and a second channel end proximate a second end of the sleeve and separated from the second end of the sleeve by a second end portion.

6. The protective sleeve of claim 2, wherein the aperture comprises a transparent or translucent material through which at least a portion of a vape fluid cartridge within the cylindrical cavity may be viewed for perception of fluid level therein.

7. The protective sleeve of claim 1, wherein the sleeve is comprised of silicone.

8. The protective sleeve of claim 1, wherein the sleeve is comprised of a material selected from amongst: urethane, polyurethane, thermoplastic, polyethylene and elastomer.

9. The protective sleeve of claim 1, wherein the sleeve is comprised of an elastic material.

10. The protective sleeve of claim 1, wherein the sleeve has a thickness of about 3 mm.

11. The protective sleeve of claim 1, wherein the sleeve has a thickness ranging from 2.5 mm to 3.5 mm.

12. The protective sleeve of claim 1, the protective sleeve further having a cylindrical exterior surface.

13. The protective sleeve of claim 12, further comprising an elevated section extending outwards from an exterior surface of the sleeve, thereby inhibiting rolling of the sleeve when placed on a flat surface.

14. The protective sleeve of claim 13, in which the elevated section forms a flat surface generally rectangular in shape.

15. The protective sleeve of claim 13, wherein the elevated section comprises ornamentation.

16. The protective sleeve of claim 1, further comprising a plurality of semi-spherical bumps formed on an outer surface thereof.

17. The protective sleeve of claim 1, further comprising an outer perimeter having a polygonal cross-section.

18. The protective sleeve of claim 1, the sleeve formed from a single continuous length of flat material wrapped in a spiral pattern to form said cylindrical internal cavity.

19. The protective sleeve of claim 18, wherein the aperture comprises a gap between adjacent portions of the length of material, through which portions of the cylindrical internal cavity may be viewed.
